(19) United States
(12) Patent Application Publication
(10) Pub. No.: US 2023/0282361 A1
(43) Pub. Date: Sep. 7, 2023

(54) INTEGRATED, MACHINE LEARNING POWERED, MEMBER-CENTRIC SOFTWARE AS A SERVICE (SAAS) ANALYTICS

(71) Applicant: Inovalon, Inc., Bowie, MD (US)

(72) Inventors: James Clement, Bowie, MD (US); Kristopher Volrath, Bowie, MD (US); Jason Astrinskiy, Bowie, MD (US); Michael Hasbany, Bowie, MD (US)

(21) Appl. No.: 18/118,001

(22) Filed: Mar. 6, 2023

Related U.S. Application Data
(60) Provisional application No. 63/317,474, filed on Mar. 7, 2022.
(60) Provisional application No. 63/317,490, filed on Mar. 7, 2022.

(51) Int. Cl. G16H 50/30; G06N 20/00; G16H 10/60

(52) U.S. Cl. CPC G16H 50/30; G06N 20/00; G16H 10/60

(57) ABSTRACT

Implementations are directed to improving healthcare services. In some aspects, a method includes receiving a plurality of requests for assessing quality of care, the plurality of requests for assessing quality of care including health data of a plurality of patients; training, a risk score machine learning (ML) model using the health data; receiving, from a user device, a request for assessing of quality of care for a particular patient, the request including the particular patient’s private health information; performing assessment of quality of care using the particular patient’s private health information; generating a risk score input from the particular patient’s private health information; generating risk scores for one or more potential health conditions for the particular patient by executing the risk score ML model using the risk score input; and providing i) the quality of care assessment result and ii) the risk scores for output on the user’s device.

### CROSS-REFERENCE TO RELATED APPLICATIONS

This application claims the benefit of U.S. Provisional Pat. Application No. 63/317,474, filed Mar. 7, 2022, and U.S. Provisional Patent Application No. 63/317,490, filed Mar. 7, 2022, which are hereby incorporated by reference in their entirety.

### TECHNICAL FIELD

This specification generally relates to integrated, machine learning powered, member-centric software as a service (SaaS) analytics platform that unifies both improving the quality of care for members and supporting risk score accuracy for health plans all while supporting adherence to applicable compliance management system (CMS) compliance guidelines.

### BACKGROUND

In a healthcare environment, a patient may visit multiple doctors and healthcare providers. These doctors and healthcare providers may not communicate with each other and may perform their own tests and medical diagnoses. The patient’s health data may be distributed at various healthcare providers.

Assessment of the quality of care a patient has received in the past can be important to improve the healthcare services and to drive healthcare costs down. For example, in assessment of the quality of care, data relating to a patient’s past medical history is mined and analyzed to determine the quality of care a patient has received. Such an assessment can indicate whether the patient has received appropriate healthcare with appropriate costs.

Further, generating risk scores for one or more potential health conditions for patients can be important to improve the healthcare services and to prevent diseases. For example, in risk score prediction, a patient’s past medical history is mined and analyzed to predict potential health conditions and a risk score that indicate the likelihood of each potential condition.

### SUMMARY

This specification is generally directed to not only performing assessment of quality of care using a patient’s aggregated universal health data, but also using the data for assessing the quality of care to train a machine learning model for predicting risk scores with high accuracy.

In one aspect, this document describes a method for both improving the quality of care for members and supporting risk score accuracy. The method includes receiving, by one or more computing devices, a plurality of requests for assessing quality of care, the plurality of requests for assessing quality of care including health data of a plurality of patients; training, a risk score machine learning (ML) model using the health data of the plurality of patients; receiving, from a user device, a request for assessing of quality of care for a particular patient, the request including the particular patient’s private health information; performing assessment of quality of care for the particular patient using the particular patient’s private health information to obtain a quality of care assessment result; generating a risk score input from the particular patient’s private health information; generating risk scores for one or more potential health conditions for the particular patient by executing the risk score ML model using the risk score input; and providing i) the quality of care assessment result and ii) the risk scores for the one or more potential health conditions of the particular patient for output on the user’s device.

Other embodiments of this aspect include corresponding computer systems, apparatus, computer program products, and computer programs recorded on one or more computer storage devices, each configured to perform the actions of the method. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

The foregoing and other embodiments can each optionally include one or more of the following features, alone or in combination. In some implementations, generating the risk score ML model can include de-identifying the health data of the plurality of patients; and training the risk score ML model using the de-identified health data of the plurality of patients. In some implementations, generating the risk score ML model can include: training the risk score ML model using all data included in the health data of the plurality of patients; or training the risk score ML model using part of the health data of the plurality of patients. In some implementations, generating the risk score input from the particular patient’s private health information can include: transforming at least a portion of the particular patient’s private health information into a different format that the risk score ML model recognizes and operates on; or aggregating the received particular patient’s private health information with incremental information obtained from a different source.

In some implementations, the method can include determining that the one or more risk scores satisfy a score threshold; and in response to determining that the one or more risk scores satisfy the threshold, recommending one or more intervention steps for the one or more potential health conditions. In some implementations, recommending the one or more intervention steps can include: determining healthcare resources, corresponding to the one or more potential health conditions, that are available in an area the particular patient selected. In some implementations, the method can include selecting the intervention steps from the available healthcare resources based on the particular patient’s personal status including financial status, personal preferences, and medical history.

Particular implementations of the subject matter described in this disclosure can be implemented so as to realize one or more of the following advantages. Assessment of quality of care can be performed for a plurality of patients in response to requests that include the health data of the plurality of patients. The health data obtained for assessing the quality of care can be further used to train a machine learning model that predicts the health risks of patients. For example, the machine learning (ML) model can predict a risk score for one or more conditions a patient might have to promote health and prevent disease. Because the quality of care assessment service is not limited to a particular disease type, the requests for quality of care assessment include health data of many different patients with various health conditions. That is the health data received for quality of care assessment is a large, diverse, and comprehensive dataset. Such a dataset can be further utilized to train a risk score ML model. The trained risk score ML model is more accurate and robust. Furthermore, since the same computing system can provide both the quality of care assessment and the risk scores, fewer requests need to be transmitted. As a result, fewer copies of the private health information of patients are transmitted and distributed, which can be helpful to protect patients’ privacy, and more efficient in managing the patients’ data by consuming less storage resources.

It is appreciated that methods and systems in accordance with the present disclosure can include any combination of the aspects and features described herein. That is, methods and systems in accordance with the present disclosure are not limited to the combinations of aspects and features specifically described herein, but also may include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A is a block diagram of an example environment for providing both quality of care assessment services and risk score services in one implementation.

FIG. 1B is a block diagram of an example environment for providing both quality of care assessment services and risk score services in an enhanced implementation

FIG. 2 is a flow diagram of an example process for providing both quality of care assessment services and risk score services.

FIG. 3 is a schematic diagram of an exemplary generic computer system.

### DETAILED DESCRIPTION

This specification generally relates to integrated, machine learning (ML) powered, member-centric software as a service (SaaS) analytics platform that unifies both improving the quality of care for members and supporting risk score accuracy for health plans all while supporting adherence to applicable compliance management system (CMS) compliance guidelines.

To provide further context, and as introduced above, doctors and healthcare providers may perform their own tests and medical diagnoses and may not communicate with each other. The patient’s health data may be distributed at the various healthcare providers. Because each healthcare provider only holds part of the patient’s data, it can be challenging for a healthcare provider to provide high-quality services. A healthcare provider may want to order a quality of care assessment for a patient. Such an assessment can help physicians to know where a patient stands while avoiding duplicated tests. Furthermore, a healthcare provider may want to order a risk score service for prevention of potential diseases. In some examples, other users, such as an insurance company, may want to order the quality of care assessment and the risk score service for a patient.

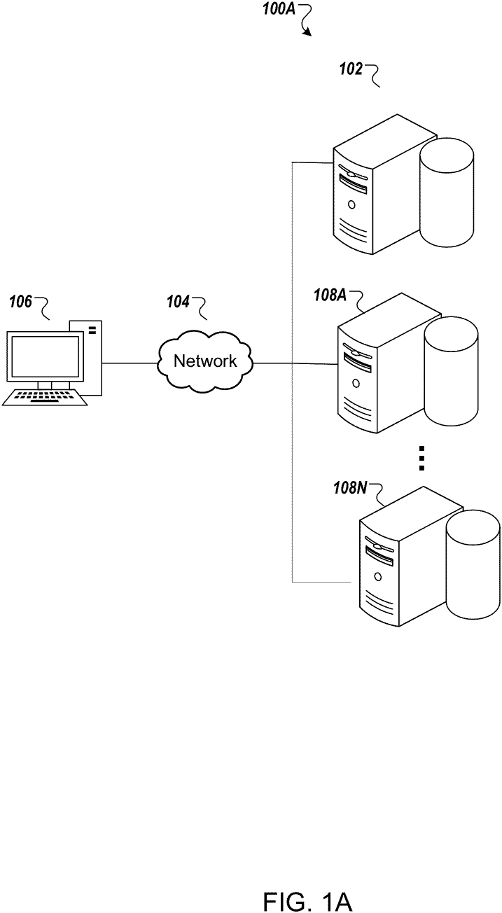

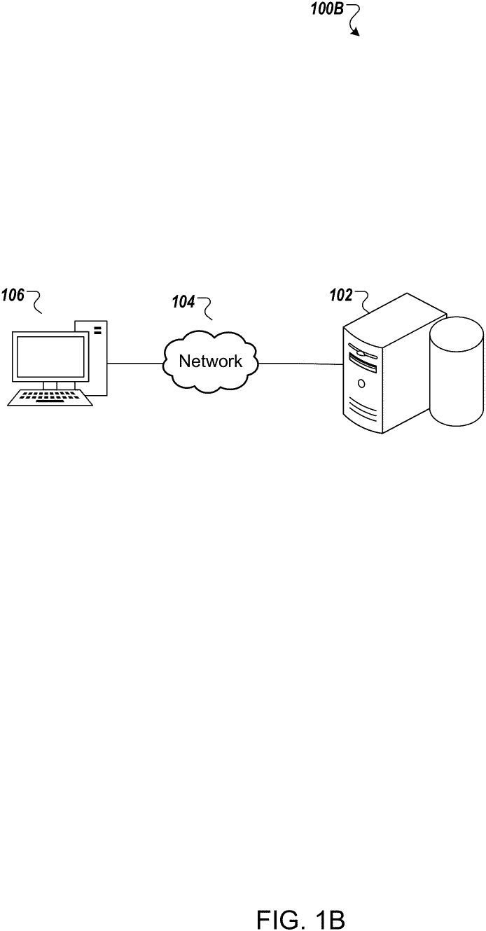

FIG. 1A is a block diagram of an example environment 100A for providing both quality of care services and risk score services in one implementation in accordance with technology described herein. The example environment 100A includes a quality of care assessment service provider 102, a plurality of risk score service providers 108A-108N (collectively referred to as 108), a user device 106, and a communication network 104. The communication network 104 can include a local area network (“LAN”), wide area network (“WAN”), the Internet, or a combination thereof.

The quality of care assessment service provider 102 can be a computing system including one or more computing devices. The quality of care assessment service provider 102 can receive, from the user device 106 over the network 104, a plurality of requests for assessing quality of care patients received. Each request can include the health data, e.g., private health information, of a patient. For example, the health data can include diagnostics, tests, physician notes, prescriptions, and any other medical history of a patient from multiple sources. The quality of care assessment service provider 102 can aggregate, analyze, and mine the patient’s health data from multiple sources to put together a comprehensive and indepth view of various facets of a patient’s medical history. This can help healthcare providers to improve the quality of healthcare services, while at the same time reducing the inefficient expenditures associated with performing unnecessary or redundant medical tests and laboratory diagnostics.

The computing system of the quality of care assessment service provider 102 can be a computing system including various functional components. The various functional components of the quality of care assessment service provider 102 may be installed on one or more computers as separate functional components or as different modules of a same functional component. For example, the various components of the quality of care assessment service provider 102 can be implemented as computer programs installed on one or more computers in one or more locations that are coupled to each through a network. In cloud-based systems for example, these components can be implemented by individual computing nodes of a distributed computing system.

In this implementation, a user of the user device 106 can submit a request for the quality of care assessment for a patient to the quality of care assessment service provider 102. The user can also submit a separate request for risk score service for the patient to a particular risk score service provider 108A. The particular risk score service provider 108A can be selected from a plurality of risk score service providers 108A-108N based on the particular type of risk score diagnostic. For example, the risk score service provider 108A may provide risk score for cardiovascular disease; and the risk score service provider 108N may provide risk scores for diabetes.

The user of the user device 106 can be a healthcare provider, an insurance company, and the like. For example, the healthcare provider may want to know where a patient stands and use the user device 106 to issue a quality of care assessment request to the quality of care assessment service provider 102. The healthcare provider may want to know the potential health conditions of a patient, and use the user device 106 to issue a risk score request to the risk score service provider 108. In some examples, other users, such as an insurance company, may want to order the quality of care assessment and the risk score service for a patient.

The user device 106 can be an electronic device that is capable of communicating over the network 104. Example user devices can include personal computers, mobile communication devices, e.g., smart phones, and other devices that can send and receive data over the network 104. The user device 106 can also include a digital assistant device. The digital assistant can be implemented in different forms of hardware devices including, a wearable device (e.g., watch or glasses), a smart phone, a speaker device, a tablet device, or another hardware device.

The plurality of risk score service providers 108 can be separate service providers, with each providing a particular type of risk score diagnostic. Each risk score service provider 108 can be a computing system including one or more computing devices. Each risk score service provider 108 can receive, from the user device 106 over the network 104, a request for risk score service for a patient. The risk score request can include the health data of the patient relevant to the particular type of risk score diagnostic. For example, the risk score service provider 108A can provide risk score for cardiovascular disease. The risk score service provider 108A can receive requests for cardiovascular disease risk score. The received requests can include patients’ health data relevant to cardiovascular disease. Each risk score service provider 108 can have its own ML model for generating the risk score for the corresponding disease type. Based on the predicted risks, one or more medical interventions can be recommended for disease prevention.

The computing system of each risk score service provider 108 can be a computing system including various functional components. The various functional components of the risk score service provider 108 may be installed on one or more computers as separate functional components or as different modules of a same functional component. For example, the various components of the risk score service provider 108 can be implemented as computer programs installed on one or more computers in one or more locations that are coupled to each through a network. In cloud-based systems for example, these components can be implemented by individual computing nodes of a distributed computing system.

The request for the quality of care assessment for the patient can include private health information of the patient. The request for risk score service for the patient can also include the private health information of the patient. In this implementation, the private health information of a patient is duplicated for multiple times and transmitted to different service providers. It can be challenging to protect the patient’s privacy. In addition, it can be inefficient to manage the patient’s data. For example, duplicated information about the patient is stored multiple times in various locations, which can result in inefficient consumption of storage and computation resources.

In this implementation, each risk score service provider may train a risk score ML model based on the patients’ data they have received in previous requests. Because each risk score service provider provides service for a particular disease type, they receive health data relevant to that particular disease type. In other words, each risk score service provider only receives a small amount of data, e.g., data relevant to a corresponding disease type. As a result, the risk score ML model may be less accurate.

FIG. 1B is a block diagram of an example environment 100B for providing both quality of care services and risk score services in an enhanced implementation in accordance with technology described here. The example environment 100B includes the quality of care assessment service provider 102, the user device 106, and the communication network 104. The communication network 104 can include a local area network (“LAN”), wide area network (“WAN”), the Internet, or a combination thereof.

In this enhanced implementation, the quality of care assessment service provider 102 can provide both the quality of care assessment service and the risk score service. More specifically, in the enhanced implementation, the quality of care service provider can utilize the large amount of health data of different patients corresponding to different diseases to train a risk score ML model. Because the quality of care assessment service is not limited to a particular disease type, the quality of care service provider can receive requests that include health data of many different patients with various health conditions. That is, the quality of care assessment service provider 102 can obtain a large, diverse, and comprehensive health dataset from the requests for assessing quality of care. The quality of care assessment service provider 102 can use or recycle such a dataset to train the risk score ML model.

In this enhanced implementation, only one copy of the patient’s private health information is transmitted to the computing system of the quality of care service provider. By using only one copy of the patient’s private health information, the quality of care service provider can provide both the quality of care results and the risk score results. Because fewer copies of the private health information are transmitted and distributed. The enhanced implementation can be helpful to protect patients’ privacy, and more efficient in managing the patients’ data by consuming less storage resources.

In this enhanced implementation, the risk score ML model can be more accurate and robust. Because the training dataset is a larger dataset compared to each risk score service provider’s dataset in FIG. 1A, the risk score ML model in this enhanced implementation can be based on more available training data and thus be more accurate. In addition, because the risk score ML model in this enhanced implementation can be based on more diverse and comprehensive dataset including different disease types, the ML model can consider the correlation of health information of various disease types. For example, by considering both the cardiovascular disease information and the diabetes information, the ML model can be more robust, sophisticated, and accurate in predicting potential health conditions. Furthermore, the risk scores are not limited to a particular type of disease. The ML model can generate the risk scores for various potential health conditions.

In this enhanced implementation, the quality of care assessment service provider 102 can receive, from the user device 106 over the network 104, a request for assessing the quality of care for a patient. The request can include the health data, e.g., private health information, of the patient. The quality of care assessment service provider 102 can perform the assessment of the quality of care by analyzing the health data of the patient including the diagnostics, tests, physician notes, prescriptions, and any other medical history of the patient. Furthermore, the quality of care assessment service provider 102 can use the patient’s health data as input to the risk score ML model to predict risk scores of various health conditions for the patient. As discussed above, the risk score ML model trained by the quality of care assessment service provider 102 is more accurate, robust, and sophisticated. The predicted risk scores are more accurate and not limited to one particular type of disease. In some implementations, the quality of care assessment service provider 102 can provide additional information on disease burden of the patient, the potential for worsening conditions, the patient’s likelihood to take actions for the care needed, potentially insurance provider custom specific measures, and the like. The quality of care assessment service provider 102 can provide the quality of care assessment result and the risk score result to the user device 106 over the network 104. In some examples, the results can be displayed on a user interface of the user device 106.

In some examples, the quality of care assessment service provider 102 can provide recommendations on intervention steps for the one or more potential health conditions indicated by the risk scores. The quality of care assessment service provider 102 can search internal or external databases (not shown) or Internet for healthcare resources corresponding to the one or more potential health conditions. In some examples, the healthcare resource may be corresponding to an area selected by the patient. The available healthcare services can be in a certain area selected by the patient. The quality of care assessment service provider 102 can recommend intervention steps based on the available healthcare resources to the patient. The recommended intervention steps can be based on the patient’s personal status including financial status, insurance plans, personal preferences, and medical history.

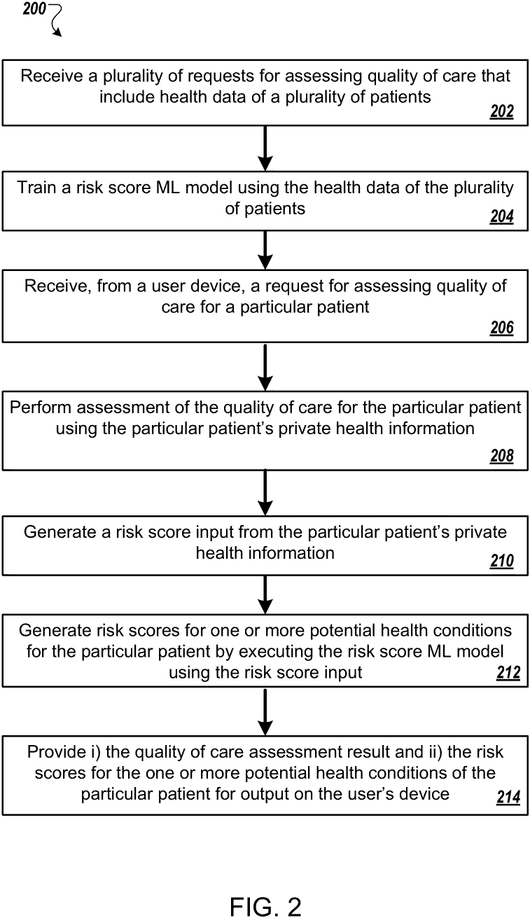

FIG. 2 is a flow diagram of an example process 200 for providing both quality of care assessment services and risk score services. In some implementations, at least a portion of the process 200 can be executed at the computing system of the quality of care assessment service provider 102.

At step 202, the computing system can receive a plurality of requests for assessing quality of care. The plurality of requests for assessing quality of care can include health data of a plurality of patients. The health data included in each request can be private health data including diagnostics, tests, physician notes, prescriptions, and any other medical history of a patient from multiple sources. The quality of care assessment service is not limited to a particular disease type, the computing system can receive many requests that include health data of many different patients with various health conditions.

At step 204, the computing system can train a risk score ML model using the health data of the plurality of patients. The computing system can obtain a large, diverse, and comprehensive health dataset from the requests for assessing quality of care. The computer system can use or recycle such a dataset to train the risk score ML model. That is the computing system can utilize the large amount of health data of different patients corresponding to different diseases to train a risk score ML model. The risk score ML model can predict risk scores for various potential health conditions using the patient’s private health data. The risk score for each health condition can indicate the probability of the patient developing the corresponding health condition.

In general, the ML model is iteratively trained, where, during an iteration, one or more parameters (e.g., weights) of the ML model are adjusted, and an output is generated based on the training data. For each iteration, a loss value is determined based on a loss function. The loss value represents a degree of accuracy of the output of the ML model. In some examples, if the loss value does not meet an expected value (e.g., is not equal to zero), parameters of the ML model are adjusted in another iteration of training. In some instances, this process is repeated until the loss value meets the expected value.

When utilizing the health data, received for quality of care assessment, to train the risk score ML model, the computing system can de-identify the health data of the plurality of patients and train the risk score ML model using the de-identified health data of the plurality of patients. For example, the computing system can remove the association between the identifying data of the patients and the health data, and use the de-identified health data for training of the risk score ML model. This can be helpful to protect the patients’ privacy.

In some examples, the computing system can train the risk score ML model using all data included in the health data of the plurality of patients. In some other examples, the computing system can train the risk score ML model using part of the health data of the plurality of patients. For example, the computing system may filter out some data included in the health data of the plurality of patients to remove bias, redundancy, or conflict.

At step 206, the computing system can receive, from a user device, a request for assessing quality of care for a particular patient. The request can include the particular patient’s private health information. For example, the patient’s private health information can include the diagnostics, tests, physician notes, prescriptions, and any other medical history of the particular patient.

In some examples, the user of the user device can be a healthcare provider, an insurance company, and the like. The healthcare provider may want to know where a patient stands and use the user device to issue a quality of care assessment request to the computing system of the quality of care assessment service provider. In some examples, other users, such as an insurance company, may want to request the quality of care assessment for a patient.

At step 208, the computing system can perform assessment of the quality of care for the particular patient using the particular patient’s private health information to obtain a quality of care assessment result. The computing system can aggregate, analyze, and mine the patient’s private health information to put together a comprehensive and in-depth view of various facets of a patient’s medical history. This can help healthcare providers to improve the quality of healthcare services, while at the same time reducing the inefficient expenditures associated with performing unnecessary or redundant medical tests and laboratory diagnostics.

At step 210, the computing system can generate a risk score input from the particular patient’s private health information. For example, the computing system can transform at least a portion of the particular patient’s private health information into a different format that the risk score ML model recognizes and operates on. In some examples, the computing system can preprocess the private health information to remove irrelevant and redundant information present or noisy and unreliable data. In some examples, the computing system can standardize the particular patient’s private health information by transforming the text of the private health information into feature vectors. The feature vectors can include numerical values representing the features of the patient’s private health information.

In another example, the computing system can aggregate the received particular patient’s private health information with incremental information obtained from a different source. The incremental information can be information supplementing the patient’s private health information, such as codes and rules on medical records and other information.

At step 212, the computing system can generate risk scores for one or more potential health conditions for the particular patent by executing the risk score ML. model using the risk score input. The one or more risk scores can indicate the likelihood or probability of the patient developing the one or more health conditions in future. In some examples, the risk scores can include a time period. For example, a risk score for diabetes can indicate that the particular patient may develop diabetes with a probability of 80% in the next 2 years. In some implementations, the computing system can provide additional information on disease burden of the patient, the potential for worsening conditions, the patient’s likelihood to take actions for the care needed, potentially insurance provider custom specific measures, and the like.

At step 214, the computing system can provide i) the quality of care assessment result and ii) the risk scores for the one or more potential health conditions of the particular patient for output on the user device. The results can be displayed on a user interface, such as a graphical user interface, of the user device. The results can be provided in an email message, a text message, a phone call, a web page, or in other forms.

Based on risk scores of the one or more health conditions, the computing system can recommend one or more intervention steps. In some implementations, the computing system can determine that the one or more risk scores satisfy a score threshold. In response to determining that the one or more risk scores satisfy the threshold, the computing system can recommend one or more intervention steps for the one or more potential health conditions. For instance, a patient’s risk score may indicate that the patient is in high risk of cardiovascular disease. The computing system can recommend intervention steps for preventing or treating cardiovascular disease.

For recommending one or more intervention steps, the computing system can determine healthcare resources, corresponding to the one or more potential health conditions. For example, the computing system can search available healthcare resources, such as experts, hospitals, treatments, for cardiovascular disease. In some examples, the healthcare resource may be available in a certain area selected by the particular patient. For example, the computing system can determine the market potential for treatments of the one or more potential health conditions in the certain area, e.g., an area the particular patient is located in or an area the particular patient selected. The computing system can search internal or external databases (not shown) or Internet for available healthcare resources.

In some examples, the computing system can recommend the intervention steps based on the available healthcare resources to the patient. The recommended intervention steps can be based on the patient’s personal status including financial status, insurance plans, personal preferences, medical history, and others. For example, the patient may be allergic to certain medication based on the patient’s medical history, the recommended indentation steps can avoid such medication. In some examples, the computing system can determine the patient’s likelihood to take actions for the care needed based on the patient’s medical history and personal preferences. The computing system can make the recommendation of the intervention steps accordingly, e.g., recommending the intervention steps that the patient is likely to take.

The order of steps in the process 200 described above is illustrative only, and the process 200 can be performed in different orders. In some implementations, the process 200 can include additional steps, fewer steps, or some of the steps can be divided into multiple steps.

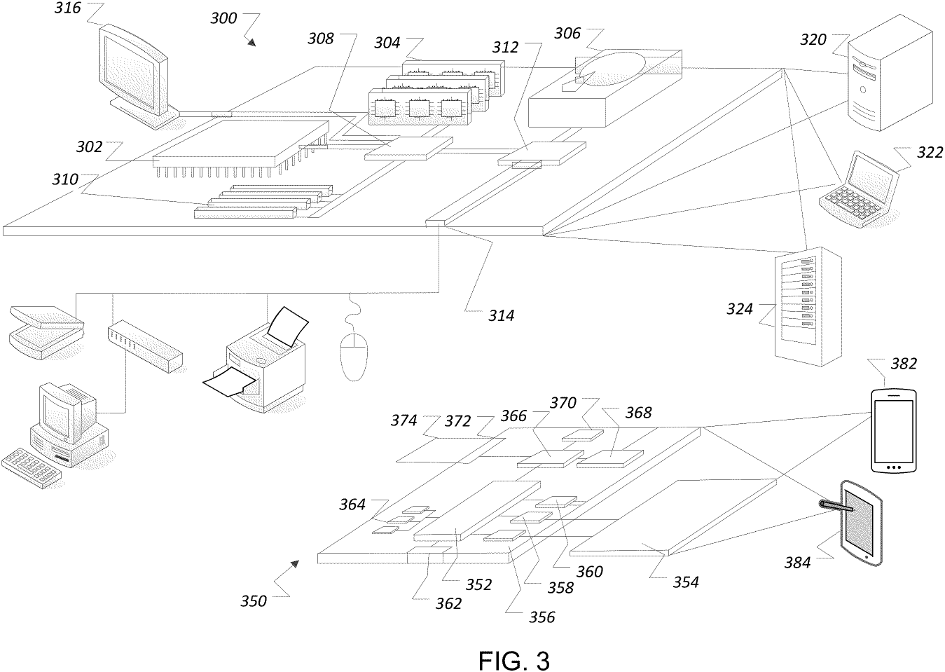

FIG. 3 shows an example of a computing device 300 and a mobile computing device 350 (also referred to herein as a wireless device) that are employed to execute implementations of the present disclosure. The computing device 300 is intended to represent various forms of digital computers, such as laptops, desktops, workstations, personal digital assistants, servers, blade servers, mainframes, and other appropriate computers. The mobile computing device 350 is intended to represent various forms of mobile devices, such as personal digital assistants, cellular telephones, smart-phones, AR devices, and other similar computing devices. The components shown here, their connections and relationships, and their functions, are meant to be examples only, and are not meant to be limiting. The computing device 300 can form at least a portion of quality of care assessment service provider 102.

The computing device 300 includes a processor 302, a memory 304, a storage device 306, a high-speed interface 308, and a low-speed interface 312. In some implementations, the high-speed interface 308 connects to the memory 304 and multiple high-speed expansion ports 310. In some implementations, the low-speed interface 312 connects to a low-speed expansion port 314 and the storage device 306. Each of the processor 302, the memory 304, the storage device 306, the high-speed interface 308, the high-speed expansion ports 310, and the low-speed interface 312, are interconnected using various buses, and may be mounted on a common motherboard or in other manners as appropriate. The processor 302 can process instructions for execution within the computing device 300, including instructions stored in the memory 304 and/or on the storage device 306 to display graphical information for a graphical user interface (GUI) on an external input/output device, such as a display 316 coupled to the high-speed interface 308. In other implementations, multiple processors and/or multiple buses may be used, as appropriate, along with multiple memories and types of memory. In addition, multiple computing devices may be connected, with each device providing portions of the necessary operations (e.g., as a server bank, a group of blade servers, or a multi-processor system).

The memory 304 stores information within the computing device 300. In some implementations, the memory 304 is a volatile memory unit or units. In some implementations, the memory 304 is a non-volatile memory unit or units. The memory 304 may also be another form of a computer-readable medium, such as a magnetic or optical disk.

The storage device 306 is capable of providing mass storage for the computing device 300. In some implementations, the storage device 306 may be or include a computer-readable medium, such as a floppy disk device, a hard disk device, an optical disk device, a tape device, a flash memory, or other similar solid-state memory device, or an array of devices, including devices in a storage area network or other configurations. Instructions can be stored in an information carrier. The instructions, when executed by one or more processing devices, such as processor 302, perform one or more methods, such as those described above. The instructions can also be stored by one or more storage devices, such as computer-readable or machine-readable mediums, such as the memory 304, the storage device 306, or memory on the processor 302.

The high-speed interface 308 manages bandwidth-intensive operations for the computing device 300, while the low-speed interface 312 manages lower bandwidth-intensive operations. Such allocation of functions is an example only. In some implementations, the high-speed interface 308 is coupled to the memory 304, the display 316 (e.g., through a graphics processor or accelerator), and to the high-speed expansion ports 310, which may accept various expansion cards. In the implementation, the low-speed interface 312 is coupled to the storage device 306 and the low-speed expansion port 314. The low-speed expansion port 314, which may include various communication ports (e.g., Universal Serial Bus (USB), Bluetooth, Ethernet, wireless Ethernet) may be coupled to one or more input/output devices. Such input/output devices may include a scanner, a printing device, or a keyboard or mouse. The input/output devices may also be coupled to the low-speed expansion port 314 through a network adapter. Such network input/output devices may include, for example, a switch or router.

The computing device 300 may be implemented in a number of different forms, as shown in the FIG. 3. For example, it may be implemented as a standard server 320, or multiple times in a group of such servers. In addition, it may be implemented in a personal computer such as a laptop computer 322. It may also be implemented as part of a rack server system 324. Alternatively, components from the computing device 300 may be combined with other components in a mobile device, such as a mobile computing device 350. Each of such devices may contain one or more of the computing device 300 and the mobile computing device 350, and an entire system may be made up of multiple computing devices communicating with each other.

The mobile computing device 350 includes a processor 352; a memory 364; an input/output device, such as a display 354; a communication interface 366; and a transceiver 368; among other components. The mobile computing device 350 may also be provided with a storage device, such as a micro-drive or other device, to provide additional storage. Each of the processor 352, the memory 364, the display 354, the communication interface 366, and the transceiver 368, are interconnected using various buses, and several of the components may be mounted on a common motherboard or in other manners as appropriate. In some implementations, the mobile computing device 350 may include a camera device(s) (not shown).

The processor 352 can execute instructions within the mobile computing device 350, including instructions stored in the memory 364. The processor 352 may be implemented as a chipset of chips that include separate and multiple analog and digital processors. For example, the processor 352 may be a Complex Instruction Set Computers (CISC) processor, a Reduced Instruction Set Computer (RISC) processor, or a Minimal Instruction Set Computer (MISC) processor. The processor 352 may provide, for example, for coordination of the other components of the mobile computing device 350, such as control of user interfaces (UIs), applications run by the mobile computing device 350, and/or wireless communication by the mobile computing device 350.

The processor 352 may communicate with a user through a control interface 358 and a display interface 356 coupled to the display 354. The display 354 may be, for example, a Thin-Film-Transistor Liquid Crystal Display (TFT) display, an Organic Light Emitting Diode (OLED) display, or other appropriate display technology. The display interface 356 may include appropriate circuitry for driving the display 354 to present graphical and other information to a user. The control interface 358 may receive commands from a user and convert them for submission to the processor 352. In addition, an external interface 362 may provide communication with the processor 352, so as to enable near area communication of the mobile computing device 350 with other devices. The external interface 362 may provide, for example, for wired communication in some implementations, or for wireless communication in other implementations, and multiple interfaces may also be used.

The memory 364 stores information within the mobile computing device 350. The memory 364 can be implemented as one or more of a computer-readable medium or media, a volatile memory unit or units, or a non-volatile memory unit or units. An expansion memory 374 may also be provided and connected to the mobile computing device 350 through an expansion interface 372, which may include, for example, a Single in Line Memory Module (SIMM) card interface. The expansion memory 374 may provide extra storage space for the mobile computing device 350, or may also store applications or other information for the mobile computing device 350. Specifically, the expansion memory 374 may include instructions to carry out or supplement the processes described above, and may include secure information also. Thus, for example, the expansion memory 374 may be provided as a security module for the mobile computing device 350, and may be programmed with instructions that permit secure use of the mobile computing device 350. In addition, secure applications may be provided via the SIMM cards, along with additional information, such as placing identifying information on the SIMM card in a non-hackable manner.

The memory may include, for example, flash memory and/or non-volatile random access memory (NVRAM), as discussed below. In some implementations, instructions are stored in an information carrier. The instructions, when executed by one or more processing devices, such as processor 352, perform one or more methods, such as those described above. The instructions can also be stored by one or more storage devices, such as one or more computer-readable or machine-readable mediums, such as the memory 364, the expansion memory 374, or memory on the processor 352. In some implementations, the instructions can be received in a propagated signal, such as, over the transceiver 368 or the external interface 362.

The mobile computing device 350 may communicate wirelessly through the communication interface 366, which may include digital signal processing circuitry where necessary. The communication interface 366 may provide for communications under various modes or protocols, such as Global System for Mobile communications (GSM) voice calls, Short Message Service (SMS), Enhanced Messaging Service (EMS), Multimedia Messaging Service (MMS) messaging, code division multiple access (CDMA), time division multiple access (TDMA), Personal Digital Cellular (PDC), Wideband Code Division Multiple Access (WCDMA), CDMA2000, General Packet Radio Service (GPRS). Such communication may occur, for example, through the transceiver 368 using a radio frequency. In addition, short-range communication, such as using a Bluetooth or Wi-Fi, may occur. In addition, a Global Positioning System (GPS) receiver module 370 may provide additional navigation- and location-related wireless data to the mobile computing device 350, which may be used as appropriate by applications running on the mobile computing device 350.

The mobile computing device 350 may also communicate audibly using an audio codec 360, which may receive spoken information from a user and convert it to usable digital information. The audio codec 360 may likewise generate audible sound for a user, such as through a speaker, e.g., in a handset of the mobile computing device 350. Such sound may include sound from voice telephone calls, may include recorded sound (e.g., voice messages, music files, etc.) and may also include sound generated by applications operating on the mobile computing device 350.

The mobile computing device 350 may be implemented in a number of different forms, as shown in FIG. 3. Other implementations may include a phone device 382 and a tablet device 384. The mobile computing device 350 may also be implemented as a component of a smart-phone, personal digital assistant, AR device, or other similar mobile device.

Computing device 300 and/or 350 can also include USB flash drives. The USB flash drives may store operating systems and other applications. The USB flash drives can include input/output components, such as a wireless transmitter or USB connector that may be inserted into a USB port of another computing device.

Although a few implementations have been described in detail above, other modifications may be made without departing from the scope of the inventive concepts described herein, and, accordingly, other implementations are within the scope of the following claims.

1. A computer-implemented method comprising:
receiving, by one or more computing devices, a plurality of requests for assessing quality of care, the plurality of requests for assessing quality of care including health data of a plurality of patients;
training, a risk score machine learning (ML) model using the health data of the plurality of patients;
receiving, from a user device, a request for assessing of quality of care for a particular patient, the request including the particular patient’s private health information;
performing assessment of quality of care for the particular patient using the particular patient’s private health information to obtain a quality of care assessment result;
generating a risk score input from the particular patient’s private health information;
generating risk scores for one or more potential health conditions for the particular patient by executing the risk score ML model using the risk score input; and
providing i) the quality of care assessment result and ii) the risk scores for the one or more potential health conditions of the particular patient for output on the user’s device.

2. The computer-implemented method of claim 1, wherein generating the risk score ML model comprises:
de-identifying the health data of the plurality of patients; and
training the risk score ML model using the de-identified health data of the plurality of patients.

3. The computer-implemented method of claim 1, wherein generating the risk score ML model comprises:
training the risk score ML model using all data included in the health data of the plurality of patients; or
training the risk score ML model using part of the health data of the plurality of patients.

4. The computer-implemented method of claim 1, wherein generating the risk score input from the particular patient’s private health information comprises:
transforming at least a portion of the particular patient’s private health information into a different format that the risk score ML model recognizes and operates on; or
aggregating the received particular patient’s private health information with incremental information obtained from a different source.

5. The computer-implemented method of claim 1, comprising:
determining that the one or more risk scores satisfy a score threshold; and
in response to determining that the one or more risk scores satisfy the threshold, recommending one or more intervention steps for the one or more potential health conditions.

6. The computer-implemented method of claim 5, wherein recommending the one or more intervention steps comprises:
determining healthcare resources, corresponding to the one or more potential health conditions, that are available in an area the particular patient selected.

7. The computer-implemented method of claim 6, comprising:
selecting the intervention steps from the available healthcare resources based on the particular patient’s personal status including financial status, personal preferences, and medical history.

8. A non-transitory computer-readable medium encoded with instructions that, when executed by one or more computers, cause the one or more computers to perform operations comprising:
receiving a plurality of requests for assessing quality of care, the plurality of requests for assessing quality of care including health data of a plurality of patients;
training, a risk score machine learning (ML) model using the health data of the plurality of patients;
receiving, from a user device, a request for assessing of quality of care for a particular patient, the request including the particular patient’s private health information;
performing assessment of quality of care for the particular patient using the particular patient’s private health information to obtain a quality of care assessment result;
generating a risk score input from the particular patient’s private health information;
generating risk scores for one or more potential health conditions for the particular patient by executing the risk score ML model using the risk score input; and
providing i) the quality of care assessment result and ii) the risk scores for the one or more potential health conditions of the particular patient for output on the user’s device.

9. The non-transitory computer-readable medium of claim 8, wherein generating the risk score ML model comprises:
de-identifying the health data of the plurality of patients; and
training the risk score ML model using the de-identified health data of the plurality of patients.

10. The non-transitory computer-readable medium of claim 8, wherein generating the risk score ML model comprises:
training the risk score ML model using all data included in the health data of the plurality of patients; or
training the risk score ML model using part of the health data of the plurality of patients.

11. The non-transitory computer-readable medium of claim 8, wherein generating the risk score input from the particular patient’s private health information comprises:
transforming at least a portion of the particular patient’s private health information into a different format that the risk score ML model recognizes and operates on; or
aggregating the received particular patient’s private health information with incremental information obtained from a different source.

12. The non-transitory computer-readable medium of claim 8, wherein the operations comprise:
determining that the one or more risk scores satisfy a score threshold; and
in response to determining that the one or more risk scores satisfy the threshold, recommending one or more intervention steps for the one or more potential health conditions.

13. The non-transitory computer-readable medium of claim 12, wherein recommending the one or more intervention steps comprises:
determining healthcare resources, corresponding to the one or more potential health conditions, that are available in an area the particular patient selected.

14. The non-transitory computer-readable medium of claim 13, wherein the operations comprise:
selecting the intervention steps from the available healthcare resources based on the particular patient’s personal status including financial status, personal preferences, and medical history.

15. A system comprising one or more computers and one or more storage devices on which are stored instructions that are operable, when executed by the one or more computers, to cause the one or more computers to perform operations comprising:
receiving a plurality of requests for assessing quality of care, the plurality of requests for assessing quality of care including health data of a plurality of patients;
training, a risk score machine learning (ML) model using the health data of the plurality of patients;
receiving, from a user device, a request for assessing of quality of care for a particular patient, the request including the particular patient’s private health information;
performing assessment of quality of care for the particular patient using the particular patient’s private health information to obtain a quality of care assessment result;
generating a risk score input from the particular patient’s private health information;
generating risk scores for one or more potential health conditions for the particular patient by executing the risk score ML model using the risk score input; and
providing i) the quality of care assessment result and ii) the risk scores for the one or more potential health conditions of the particular patient for output on the user’s device.

16. The system of claim 15, wherein generating the risk score ML model comprises:
de-identifying the health data of the plurality of patients; and
training the risk score ML model using the de-identified health data of the plurality of patients.

17. The system of claim 15, wherein generating the risk score ML model comprises:
training the risk score ML model using all data included in the health data of the plurality of patients; or
training the risk score ML model using part of the health data of the plurality of patients.

18. The system of claim 15, wherein generating the risk score input from the particular patient’s private health information comprises:
transforming at least a portion of the particular patient’s private health information into a different format that the risk score ML model recognizes and operates on; or
aggregating the received particular patient’s private health information with incremental information obtained from a different source.

19. The system of claim 15, wherein the operations comprise:
determining that the one or more risk scores satisfy a score threshold; and
in response to determining that the one or more risk scores satisfy the threshold, recommending one or more intervention steps for the one or more potential health conditions.

20. The system of claim 19, wherein recommending the one or more intervention steps comprises:
determining healthcare resources, corresponding to the one or more potential health conditions, that are available in an area the particular patient selected.
